(19) United States
(12) Patent Application Publication
(10) Pub. No.: US 2021/0116458 A1
(43) Pub. Date: Apr. 22, 2021

(54) METHODS AND PHARMACEUTICAL COMPOSITIONS FOR INHIBITING T CELL PROLIFERATION IN A SUBJECT IN NEED THEREOF

(71) Applicants: [NSERM (INSTITUT NATIONAL DE LA SANTE ET DE LA RECHERCHE MEDICALE, Paris (FR); FONDATION IMAGINE, Paris (FR); UNIVERSITE PARIS DESCARTES, Paris (FR); ASSISTANCE PUBLIQUE-HOPITAUX DE PARIS (APHP), Paris (FR)

(72) Inventors: Sylvain LATOUR, Paris (FR); Alain FISCHER, Paris (FR); Sarah WINTER, Paris (FR)

(21) Appl. No.: 16/498,747

(22) PCT Filed: Mar. 26, 2018
(86) PCT No.: PCT/EP2018/057634

§ 371 Date: Sep. 27, 2019

(30) Foreign Application Priority Data
Mar. 27, 2017 (EP) 17305345.5

(51) Int. Cl. G01N 33/68

(52) U.S. Cl. CPC G01N 33/68

(57) ABSTRACT

The inventors report two siblings presenting recurrent EBV infection and Hodgkin lymphoma caused by a homozygous loss-of-function mutation in RASGRP1, a T-cell specific nucleotide exchange factor (GEF) known to activate the RAS-induced MAPK/ERK kinases pathway. In response to TCR stimulation, RASGRP1-deficient T cells exhibited defective ERK kinases activation and impaired proliferation that was restored by expression of wild-type RASGRP1. Thus, these results identify a novel primary immunodeficiency that highlights T-cell proliferation and offers the opportunity to develop RASGRP1 inhibitor for inhibiting T cell proliferation in a subject in need thereof.

### FIELD OF THE INVENTION

The present invention relates to methods and pharmaceutical compositions for inhibiting T cell proliferation in a subject in need thereof.

### BACKGROUND OF THE INVENTION

T cell proliferation is the normal component of the immune reaction toward an antigen (e.g. a pathogen antigen). For instance, expansion of antigen-specific T-lymphocytes is a key component of adaptive immune responses. During anti-viral responses, expansion of T cells is crucial for an efficient cytotoxicity response towards infected cells. This is particularly the case during Epstein Barr virus infection, in which massive proliferation of specific-CD8+ T cells is necessary to suppress and eliminate EBV-infected B cells that strongly proliferate and may ultimately undergo transformation into lymphoma (Hislop and Taylor, 2015; Taylor et al., 2015). Several primary immune deficiencies including defects in CTPS1, MAGT1, ITK and CD27 are associated with a high susceptibility to EBV infection leading to lymphoproliferative disorders (LPD) such as non-malignant B-cell proliferation and Hodgkin and non-Hodgkin lymphomas (Cohen, 2015; Veillette et al., 2013). In addition, inherited immunodeficiencies associated with impaired cytotoxicity including defects in SH2D1A, and in components of cell lytic granule machinery such as MUNC18-2 and RAB27A, cause virus-associated hemophagocytic syndrome (VAHS) or hemophagocytic lymphohistiocytosis (HLH) upon EBV infection (Cohen, 2015). Studies of these molecular defects have highlighted two critical steps in the induction of T cell response to EBV, i.e. cell expansion and cytotoxic effector functions that depend on distinct molecules/pathways. The importance of T-cell expansion is exemplified by the CTPS1 deficiency (Martin et al., 2014). CTPS1-deficient patients exhibit early onset severe chronic EBV infections including lymphoma, as well as varicella zoster virus (VZV) infection. CTPS1 is a CTP synthetase involved in the de novo synthesis of the CTP nucleotide, a precursor of the nucleic acids metabolism. CTPS1 is rapidly upregulated in activated T cells in response to TCR stimulation. In the absence of CTPS1, the capacity of activated T cells to proliferate is impaired. Thus, CTPS1 activity is necessary for sustained proliferation of activated T cells during the immune response, which is particularly intensified in response to EBV. In this context, WO2014170435 disclosed the use of a CTPS1 inhibitor for inhibiting lymphocyte proliferation in a subject in need thereof.

### SUMMARY OF THE INVENTION

The present invention relates to methods and pharmaceutical compositions for inhibiting T cell proliferation in a subject in need thereof. In particular, the present invention is defined by the claims.

### DETAILED DESCRIPTION OF THE INVENTION

T cell proliferation is the normal component of the immune reaction toward an antigen (e.g. a pathogen antigen). However in certain circumstances T cell proliferation appears deleterious. In this context, the inventors report two siblings presenting recurrent EBV infection and Hodgkin lymphoma caused by a homozygous loss-of-function mutation in RASGRP1, a T-cell specific nucleotide exchange factor (GEF) known to activate the RAS-induced MAPK/ERK kinases pathway. In response to TCR stimulation, RASGRP1-deficient T cells exhibited defective ERK kinases activation and impaired proliferation that was restored by expression of wild-type RASGRP1. Importantly, expression of CTPS1 and PCNA, a factor involved in DNA replication were found to be defective in RASGRP1-deficient T cells, revealing the crucial role of RASGRP1-ERK pathway in the up regulation of genes required for T-cell proliferation. Thus, these results identify a novel primary immunodeficiency that highlights T-cell proliferation and offers the opportunity to develop RASGRP1 inhibitor for inhibiting T cell proliferation in a subject in need thereof.

Accordingly, the first object of the present invention relates to a method for inhibiting T cell proliferation in a subject in need thereof comprising administering to the subject a therapeutically effective amount of a RASGRP1 inhibitor.

In some embodiments, the subject suffers from a T cell lymphoproliferative disease which can include but without limitation: lymphoblastic lymphomas in which the malignancy occurs in primitive lymphoid progenitors from the thymus; mature or peripheral T cell neoplasms, including T cell prolymphocytic leukemia, T-cell granular lymphocytic leukemia, aggressive NK-cell leukemia, cutaneous T cell lymphoma (Mycosis fungoides/Sezary syndrome), anaplastic large cell lymphoma, T cell type, enteropathy-type T cell lymphoma, Adult T-cell leukemia/lymphoma including those associated with HTLV-1, and angioimmunoblastic T cell lymphoma, and subcutaneous panniculitic T cell lymphoma; and peripheral T cell lymphomas that initially involve a lymph node paracortex and never grow into a true follicular pattern. In some embodiments, the subject suffers from a T precursor acute lymphoblastic leukemia (T-ALL). T cell precursor acute lymphoblastic leukemia includes ALL-L1 and ALL-L2 (done according to the French-American-British (FAB) classification). As used herein , the term “Peripheral T-Cell Lymphoma (PTCL-NOS),” as used herein, means a group of diseases that do not fit into any of the other subtypes of PTCL. PTCL-NOS is the most common subtype, making up about one quarter of all diagnosed PTCLs. It is also the most common of all the T-cell lymphomas. The term PTCL can be confusing as it can refer to the entire spectrum of mature T-cell lymphomas or sometimes to this specific subtype, PTCL-NOS, only. Although most patients with PTCL-NOS present with lymph node involvement, sites outside the lymph nodes, such as the liver, bone marrow, gastrointestinal tract and skin, may also be involved. As used herein, the term “Anaplastic Large-Cell Lymphoma (ALCL),” as used herein, means a rare type of aggressive T-cell lymphoma comprising only 3 percent of all lymphomas in adults (about 15 percent to 20 percent of all PTCLs) and between 10 percent and 30 percent of all lymphomas in children. ALCL can appear in the skin or in other organs throughout the body (systemic ALCL). As used herein, the term “Angioimmunoblastic T-Cell Lymphoma (AITL),” as used herein, means an often fast-growing T-cell lymphoma that accounts for between 1 percent and 2 percent of all NHL cases (about 15 percent to 20 percent of all PTCLs) in the United States. As used herein, the term “Enteropathy-Type T-Cell Lymphoma,” means an extremely rare subtype that appears in the intestines and is strongly associated with celiac disease. As used herein the term “Cutaneous T-cell Lymphomas (CTCL)” means a group of lymphomas that originate in the skin. CTCLs are a subset of PTCL as they are lymphomas of mature T-cells. However, these lymphomas are generally less aggressive, have a different prognosis, and have different treatment approaches than the aggressive PTCLs. Mycosis fungoides is the most common type of cutaneous T-cell lymphoma. It is generally a slow-growing cancer that starts in the skin, appearing as a scaly, red rash in areas of the body that are not usually exposed to the sun. Sezary Syndrome is an advanced, variant form of mycosis fungoides, and affects both the skin and the peripheral blood. It can cause widespread itching, reddening and peeling of the skin as well as skin tumors. In some embodiments, the subject suffers from an autoimmune inflammatory disease.

In particular, the subject suffers from an autoimmune inflammatory disease selected from the group consisting of arthritis, rheumatoid arthritis, acute arthritis, chronic rheumatoid arthritis, gouty arthritis, acute gouty arthritis, chronic inflammatory arthritis, degenerative arthritis, infectious arthritis, Lyme arthritis, proliferative arthritis, psoriatic arthritis, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, dermatitis including contact dermatitis, chronic contact dermatitis, allergic dermatitis, allergic contact dermatitis, dermatitis herpetiformis, and atopic dermatitis, x-linked hyper IgM syndrome, urticaria such as chronic allergic urticaria and chronic idiopathic urticaria, including chronic autoimmune urticaria, polymyositis/dermatomyositis, juvenile dermatomyositis, toxic epidermal necrolysis, scleroderma, systemic scleroderma, sclerosis, systemic sclerosis, multiple sclerosis (MS), spino-optical MS, primary progressive MS (PPMS), relapsing remitting MS (RRMS), progressive systemic sclerosis, atherosclerosis, arteriosclerosis, sclerosis disseminata, and ataxic sclerosis, inflammatory bowel disease (IBD), Crohn's disease, colitis, ulcerative colitis, colitis ulcerosa, microscopic colitis, collagenous colitis, colitis polyposa, necrotizing enterocolitis, transmural colitis, autoimmune inflammatory bowel disease, pyoderma gangrenosum, erythema nodosum, primary sclerosing cholangitis, episcleritis, respiratory distress syndrome, adult or acute respiratory distress syndrome (ARDS), meningitis, inflammation of all or part of the uvea, iritis, choroiditis, an autoimmune hematological disorder, rheumatoid spondylitis, sudden hearing loss, IgE-mediated diseases such as anaphylaxis and allergic and atopic rhinitis, encephalitis, Rasmussen's encephalitis, limbic and/or brainstem encephalitis, uveitis, anterior uveitis, acute anterior uveitis, granulomatous uveitis, nongranulomatous uveitis, phacoantigenic uveitis, posterior uveitis, autoimmune uveitis, glomerulonephritis (GN), idiopathic membranous GN or idiopathic membranous nephropathy, membrano- or membranous proliferative GN (MPGN), rapidly progressive GN, allergic conditions, autoimmune myocarditis, leukocyte adhesion deficiency, systemic lupus erythematosus (SLE) or systemic lupus erythematodes such as cutaneous SLE, subacute cutaneous lupus erythematosus, neonatal lupus syndrome (NLE), lupus erythematosus disseminatus, lupus (including nephritis, cerebritis, pediatric, non-renal, extra-renal, discoid, alopecia), juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, immune responses associated with acute and delayed hypersensitivity mediated by cytokines and T-lymphocytes, tuberculosis, sarcoidosis, granulomatosis, lymphomatoid granulomatosis, Wegener's granulomatosis, agranulocytosis, vasculitides, including vasculitis, large vessel vasculitis, polymyalgia rheumatica, giant cell (Takayasu's) arteritis, medium vessel vasculitis, Kawasaki's disease, polyarteritis nodosa, microscopic polyarteritis, CNS vasculitis, necrotizing, cutaneous, hypersensitivity vasculitis, systemic necrotizing vasculitis, and ANCA-associated vasculitis, such as Churg-Strauss vasculitis or syndrome (CSS), temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Bechet's or Behcet's disease, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus, optionally pemphigus vulgaris, pemphigus foliaceus, pemphigus mucus-membrane pemphigoid, pemphigus erythematosus, autoimmune polyendocrinopathies, Reiter's disease or syndrome, immune complex nephritis, antibody-mediated nephritis, neuromyelitis optica, polyneuropathies, chronic neuropathy, IgM polyneuropathies, IgM-mediated neuropathy, thrombocytopenia, thrombotic thrombocytopenic purpura (TTP), idiopathic thrombocytopenic purpura (ITP), autoimmune orchitis and oophoritis, primary hypothyroidism, hypoparathyroidism, autoimmune thyroiditis, Hashimoto's disease, chronic thyroiditis (Hashimoto's thyroiditis); subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis, allergic encephalomyelitis, experimental allergic encephalomyelitis (EAE), myasthenia gravis, thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant cell hepatitis, chronic active hepatitis or autoimmune chronic active hepatitis, lymphoid interstitial pneumonitis, bronchiolitis obliterans (non-transplant) vs NSIP, Guillain-Barre syndrome, Berger's disease (IgA nephropathy), idiopathic IgA nephropathy, linear IgA dermatosis, primary biliary cirrhosis, pneumonocirrhosis, autoimmune enteropathy syndrome, Celiac disease, Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AGED), autoimmune hearing loss, opsoclonus myoclonus syndrome (OMS), polychondritis such as refractory or relapsed polychondritis, pulmonary alveolar proteinosis, amyloidosis, scleritis, a non-cancerous lymphocytosis, a primary lymphocytosis, which includes monoclonal B cell lymphocytosis, optionally benign monoclonal gammopathy or monoclonal gammopathy of undetermined significance, MGUS, peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases, diabetic nephropathy, Dressler's syndrome, alopecia greata, CREST syndrome (calcinosis, Raynaud's phenomenon, esophageal dysmotility, sclerodactyl), and telangiectasia), male and female autoimmune infertility, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis, or Fuch's cyclitis, Henoch-Schonlein purpura, human immunodeficiency virus (HIV) infection, echovirus infection, cardiomyopathy, Alzheimer's disease, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, endocrine ophthamopathy, chronic hypersensitivity pneumonitis, keratoconjunctivitis sicca, epidemic keratoconjunctivitis, idiopathic nephritic syndrome, minimal change nephropathy, benign familial and ischemia-reperfusion injury, retinal autoimmunity, joint inflammation, bronchitis, chronic obstructive airway disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired splenic atrophy, infertility due to antispermatozoan antobodies, non-malignant thymoma, vitiligo, SCID and Epstein-Barr virus-associated diseases, acquired immune deficiency syndrome (AIDS), parasitic diseases such as Lesihmania, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, peripheral neuropathy, autoimmune polyglandular syndrome type I, adult-onset idiopathic hypoparathyroidism (AOIH), alopecia totalis, dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia, autoimmune disorders associated with collagen disease, rheumatism, neurological disease, ischemic re-perfusion disorder, reduction in blood pressure response, vascular dysfunction, antgiectasis, tissue injury, cardiovascular ischemia, hyperalgesia, cerebral ischemia, and disease accompanying vascularization, allergic hypersensitivity disorders, glomerulonephritides, reperfusion injury, reperfusion injury of myocardial or other tissues, dermatoses with acute inflammatory components, acute purulent meningitis or other central nervous system inflammatory disorders, ocular and orbital inflammatory disorders, granulocyte transfusion-associated syndromes, cytokine-induced toxicity, acute serious inflammation, chronic intractable inflammation, pyelitis, pneumonocirrhosis, diabetic retinopathy, diabetic large-artery disorder, endarterial hyperplasia, peptic ulcer, valvulitis, and endometriosis.

In some embodiments, the autoimmune inflammatory disease is secondary to therapeutic treatment, in particular a treatment with an immune checkpoint inhibitor. As used herein, the term “immune checkpoint inhibitor” has its general meaning in the art and refers to any compound inhibiting the function of an immune inhibitory checkpoint protein. Inhibition includes reduction of function and full blockade. Preferred immune checkpoint inhibitors are antibodies that specifically recognize immune checkpoint proteins. In some embodiments, the immune checkpoint inhibitor is an antibody selected from the group consisting of anti-CTLA4 antibodies, anti-PD-1 antibodies, anti-PD-L1 antibodies, anti-PD-L2 antibodies anti-TIM-3 antibodies, anti-LAG3 antibodies, anti-B7H3 antibodies, anti-B7H4 antibodies, anti-BTLA antibodies, and anti-B7H6 antibodies.

In some embodiments, the subject suffers from an allergic disorder. As used herein, “allergic disorder” refers to any disorder resulting from antigen activation of mast cells that results in an “allergic reaction” or state of hypersensitivity and influx of inflammatory and immune cells. Those disorders include without limitation: systemic allergic reactions, systemic anaphylaxis or hypersensitivity responses, anaphylactic shock, drug allergies, and insect sting allergies; respiratory allergic diseases, such asthma, hypersensitivity lung diseases, hypersensitivity pneumonitis and interstitial lung diseases (ILD) (e.g. idiopathic pulmonary fibrosis, ILD associated with rheumatoid arthritis, or other autoimmune conditions); rhinitis, hay fever, conjunctivitis, allergic rhinoconjunctivitis and vaginitis; skin and dermatological disorders, including psoriasis and inflammatory dermatoses, such as dermatitis, eczema, atopic dermatitis, allergic contact dermatitis, dermatitis herpetiforms, linear IgA disease, acute and chronic urticaria and scleroderma; vasculitis (e.g. necrotizing, cutaneous, and hypersensitivity vasculitis); spondyloarthropathies; and intestinal reactions of the gastrointestinal system (e.g., inflammatory bowel diseases such as Crohn's disease, ulcerative colitis, ileitis, enteritis, nontropical sprue and celiac disease). In some embodiments, the subject suffers from asthma. As used herein, the term “asthma” refers to an inflammatory disease of the respiratory airways that is characterized by airway obstruction, wheezing, and shortness of breath.

Accordingly, the method of the present invention is particular suitable for the treatment of T cell lymphoma, autoimmune inflammatory diseases, and allergic disorders. As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

In some embodiments, the subject is a transplanted subject. Typically the subject may have been transplanted with a graft selected from the group consisting of heart, kidney, lung, liver, pancreas, pancreatic islets, brain tissue, stomach, large intestine, small intestine, cornea, skin, trachea, bone, bone marrow, muscle, or bladder. The method of the invention is indeed particularly suitable for preventing or suppressing an immune response associated with rejection of a donor tissue, cell, graft, or organ transplant by a recipient subject. Graft-related diseases or disorders include graft versus host disease (GVDH), such as associated with bone marrow transplantation, and immune disorders resulting from or associated with rejection of organ, tissue, or cell graft transplantation (e.g., tissue or cell allografts or xenografts), including, e.g., grafts of skin, muscle, neurons, islets, organs, parenchymal cells of the liver, etc. With regard to a donor tissue, cell, graft or solid organ transplant in a recipient subject, it is believed that RASGRP1 inhibitor according to the invention may be effective in preventing acute rejection of such transplant in the recipient and/or for long-term maintenance therapy to prevent rejection of such transplant in the recipient (e.g., inhibiting rejection of insulin-producing islet cell transplant from a donor in the subject recipient suffering from diabetes). Thus the method of the invention is useful for preventing Host-Versus-Graft-Disease (HVGD) and Graft-Versus-Host-Disease (GVHD). The RASGRP1 inhibitor may be administered to the subject before and/or after transplantation (e.g., at least one day before transplantation, from one to five days after transplantation, etc.). In some embodiments, the RASGRP1 inhibitor may be administered to the subject on a periodic basis before and/or after transplantation.

As used herein the term “RASGRP1” has its general meaning in the art and refers to AS guanyl releasing protein 1 encode by RASGRP1 gene (Gene ID n° 10125). The term is also known as RASGRP; hRasGRP1; CALDAG-GEFI; and CALDAG-GEFII. The protein is characterized by the presence of a Ras superfamily guanine nucleotide exchange factor (GEF) domain. It functions as a diacylglycerol (DAG)-regulated nucleotide exchange factor specifically activating Ras through the exchange of bound GDP for GTP. It activates the Erk/MAP kinase cascade. Examplary nucleic and amino acid sequences are represented by the NCBI reference sequences NM 005739.3 and NP 005730.2 respectively.

As used herein, the term “RASGRP1 inhibitor” refers to any compound which has the ability of reducing or suppressing the activity or expression of RASGRP1. Typically the RASGRP1 inhibitor can act directly on the activity by binding to the protein, or can act indirectly on the activity by reducing or inhibiting the expression of the enzyme. Thus RASGRP1 inhibitors encompass inhibitor of RASGRP1 expression. For example, RASGRP1 inhibitors also include any compound that can compete with the substrate of RASGRP1 to the corresponding catalytic domains. Typically, said inhibitor is a small organic molecule or a biological molecule (e.g. peptides, aptamers . . . ).

An “inhibitor of expression” refers to a natural or synthetic compound that has a biological effect to inhibit the expression of a gene. In a preferred embodiment of the invention, said inhibitor of gene expression is a siRNA, an antisense oligonucleotide or a ribozyme. For example, anti-sense oligonucleotides, including anti-sense RNA molecules and anti-sense DNA molecules, would act to directly block the translation of RASGRP1 mRNA by binding thereto and thus preventing protein translation or increasing mRNA degradation, thus decreasing the level of RASGRP1, and thus activity, in a cell. For example, antisense oligonucleotides of at least about 15 bases and complementary to unique regions of the mRNA transcript sequence encoding RASGRP1 can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. RASGRP1 gene expression can be reduced by contacting a patient or cell with a small double stranded RNA (dsRNA), or a vector or construct causing the production of a small double stranded RNA, such that RASGRP1 gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing RASGRP1. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art. In some embodiments, the inhibitor of expression is an endonuclease. The term “endonuclease” refers to enzymes that cleave the phosphodiester bond within a polynucleotide chain. Some, such as Deoxyribonuclease I, cut DNA relatively nonspecifically (without regard to sequence), while many, typically called restriction endonucleases or restriction enzymes, and cleave only at very specific nucleotide sequences. The mechanism behind endonuclease-based genome inactivating generally requires a first step of DNA single or double strand break, which can then trigger two distinct cellular mechanisms for DNA repair, which can be exploited for DNA inactivating: the error prone non homologous end joining (NHEJ) and the high-fidelity homology-directed repair (HDR). In a particular embodiment, the endonuclease is CRISPR-Css. As used herein, the term “CRISPR-Cas” has its general meaning in the art and refers to clustered regularly interspaced short palindromic repeats associated which are the segments of prokaryotic DNA containing short repetitions of base sequences. In some embodiment, the endonuclease is CRISPR-cas9 which is from Streptococcus pyogenes. The CRISPR/Cas9 system has been described in U.S. Pat. No. 8,697,359 B1 and US 2014/0068797. In some embodiment, the endonuclease is CRISPR-Cpf1 which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

A “therapeutically effective amount” refers to an amount effective, at dosages and for periods of time necessary, to achieve a desired therapeutic result. A therapeutically effective amount of the antibody of the present invention may vary according to factors such as the disease state, age, sex, and weight of the individual, and the ability of the antibody of the present invention to elicit a desired response in the individual. A therapeutically effective amount is also one in which any toxic or detrimental effects of the antibody or antibody portion are outweighed by the therapeutically beneficial effects. The efficient dosages and dosage regimens for the antibody of the present invention depend on the disease or condition to be treated and may be determined by the persons skilled in the art. A physician having ordinary skill in the art may readily determine and prescribe the effective amount of the pharmaceutical composition required. For example, the physician could start doses of the antibody of the present invention employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above. An exemplary, non-limiting range for a therapeutically effective amount of the inhibitor is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation.

Typically, the inhibitor of the present invention is administered to the patient in the form of a pharmaceutical composition which comprises a pharmaceutically acceptable carrier. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene- block polymers, polyethylene glycol and wool fat. For use in administration to a patient, the composition will be formulated for administration to the patient. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono-or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m² and 500 mg/m². However, it will be appreciated that these schedules are exemplary and that an optimal schedule and regimen can be adapted taking into account the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g. 1 ml for intramuscular), and between about 1 ng to about 100 mg, e.g. about 50 ng to about 30 mg or more preferably, about 5 mg to about 25 mg, of the inhibitor of the invention.

A further aspect of the invention relates to a method for screening a plurality of test substances useful for inhibiting T cell proliferation in a subject in need thereof comprising the steps consisting of i) testing each of the test substances for its ability to inhibit RASGRP1 activity or expression and ii) identifying the test substance which inhibits RASGRP1 activity or expression thereby to identify a test substance useful for inhibiting T cell proliferation in a subject in need thereof.

Any assay well known in the art may be used for testing the ability of test substance to inhibit RASGRP1 activity. In particular the assay may consist in the use of purified substrate RAS and then in determining the GTPase activity of RAS in the presence of RASGRP1 by the measurement of phosphates liberated from RAS. It is merely required that the substrate is appropriately labelled so that its conversion can be detected by detecting the label in a product of the biosynthetic pathway. The substrate is preferably loaded with a labelled GTP. Typically, the labelled substrate may be non-radioactive or radioactive. For example, in case of a non-radioactive substrate, P³² labelled or deuterium-labelled substrates may be. For example, in case of radioactive substrates, Gamma-P³²-labelled substrates are preferred. Typically, the labeled substrates may be added as aqueous solution with RASGRP1. The concentration of the substrates in the aqueous solution may be 1 μM to 1 mM. In case of P³²-labelled substrates the radioactivity is preferably at least 0.1μ Ci to 1μ Ci. The labelling with 32-Phosphate may be single whereby any one of the C-positions may be labelled. Alternatively, the substrates may be multiply labelled, such as dual, triple, quadruple or quintuple. The total C-labelling is particularly preferred in case of 13-carbon labelling. The labelling with deuterium or tritium may be single or multiple. Typically, the labelled substrates may be prepared enzymatically or chemically. The substrate, the test substance and the enzyme are typically incubated in time sufficient for allowing the enzymatic conversion. It is then possible to separate from the solution the product obtained by the conservation of the substrate, by HPLC, thin layer chromatography or the like. In case of radioactive labelling, the determination of labelled product may be effected by a scintillation counter, by a phosphorimager, by a radio thin layer counter or by a radio detector in combination with a chromatographic column. Typically, a connection of the HPLC to a Flow Scintillation Analyzer (Radiomatic 150 TR, Packard) made it possible to check the radioactivity in the chromatographic peaks. For radioactivity measurements, the whole sample was usually loaded onto the column. The labeled products were quantified by measuring the peak heights and comparing them to a standard curve. In case of non-radioactive labelling, the determination may be effected conventionally by NMR spectroscopy (e.g. ¹³C-NMR) or mass spectroscopy (e.g. HPLC-MS or GC-MS). A test substance is considered as a RASGRP1 inhibitor when the amount of the labeled product is lower than the amount of the labeled product determined in the absence of the test substance.

A variety of cells may be used in the in vitro assays. Typically the cell is a T cell which expresses naturally RASGRP1. In some embodiments, a broad variety of host-expression vector systems may be utilized to express RASGRP1 in a cell of interest. These include, but are not limited to, mammalian cell systems such as human cell lines. The mammalian cell systems may harbour recombinant expression constructs containing promoters derived from the genome of mammalian cells or from mammalian viruses (e.g., the adenovirus late promoter or the vaccine virus 7.5K promoter). DNA encoding proteins to be assayed (i.e. RASGRP1) can be transiently or stably expressed in the cell lines by several methods known in the art, such as, calcium phosphate-mediated, DEAE-dextran mediated, liposomal-mediated, viral-mediated, electroporation-mediated and microinjection delivery. Each of these methods may require optimization of assorted experimental parameters depending on the DNA, cell line, and the type of assay to be subsequently employed. In addition native cell lines that naturally carry and express the nucleic acid sequences for the target protein may be used.

In well-known assay in the art may also be used for determining whether a test substance is able to inhibit the expression of RASGRP1. Typically, a population of cells expressing RASGRP1 is cultured in the presence of the test substance and the expression level of RASGRP1 is then determined and compared to the level determined in the absence of the test substance. It is concluded that the test substance is a RASGRP1 inhibitor when the level of RASGRP1 expression determined in the presence of the test substance is lower than the level of RASGRP1 expression determined in the absence of the test substance. The determination of the expression level of a gene can be performed by a variety of techniques. Generally, the expression level as determined is a relative expression level. More preferably, the determination comprises contacting the sample with selective reagents such as probes, primers or ligands, and thereby detecting the presence, or measuring the amount, of polypeptide or nucleic acids of interest originally in the sample. Contacting may be performed in any suitable device, such as a plate, microtiter dish, test tube, well, glass, column, and so forth In specific embodiments, the contacting is performed on a substrate coated with the reagent, such as a nucleic acid array or a specific ligand array. The substrate may be a solid or semi-solid substrate such as any suitable support comprising glass, plastic, nylon, paper, metal, polymers and the like. The substrate may be of various forms and sizes, such as a slide, a membrane, a bead, a column, a gel, etc. The contacting may be made under any condition suitable for a detectable complex, such as a nucleic acid hybrid or an antibody-antigen complex, to be formed between the reagent and the nucleic acids or polypeptides of the sample. In a preferred embodiment, the expression level may be determined by determining the quantity of mRNA. Methods for determining the quantity of mRNA are well known in the art. For example the nucleic acid contained in the samples (e.g., cell or tissue prepared from the subject) is first extracted according to standard methods, for example using lytic enzymes or chemical solutions or extracted by nucleic-acid-binding resins following the manufacturer's instructions. The extracted mRNA is then detected by hybridization (e.g., Northern blot analysis) and/or amplification (e.g., RT-PCR). Preferably quantitative or semi-quantitative RT-PCR is preferred. Real-time quantitative or semi-quantitative RT-PCR is particularly advantageous. Other methods for determining the expression level of said genes include the determination of the quantity of proteins encoded by said genes. The presence of the protein can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith.

Typically, the test substance of may be selected from the group consisting of peptides, peptidomimetics, small organic molecules, antibodies, aptamers or nucleic acids. For example the test substance according to the invention may be selected from a library of compounds previously synthesized, or a library of compounds for which the structure is determined in a database, or from a library of compounds that have been synthesized de novo. In a particular embodiment, the test substance may be selected form small organic molecules. As used herein, the term “small organic molecule” refers to a molecule of size comparable to those organic molecules generally sued in pharmaceuticals. The term excludes biological macromolecules (e.g.; proteins, nucleic acids, etc.); preferred small organic molecules range in size up to 2000 Da, and most preferably up to about 1000 Da.

The screening methods of the invention are very simple. It can be performed with a large number of test substances, serially or in parallel. The method can be readily adapted to robotics.

For example, the above assays may be performed using high throughput screening techniques for identifying test substances for developing drugs that may be useful to the treatment or prevention of an inflammatory bowel disease. High throughput screening techniques may be carried out using multi-well plates (e.g., 96-, 389-, or 1536-well plates), in order to carry out multiple assays using an automated robotic system. Thus, large libraries of test substances may be assayed in a highly efficient manner. A preferred strategy for identifying test substances starts with cultured cells transfected with a reporter gene fused to the promoter of any gene that is activated by the stress response pathway. More particularly, stably-transfected cells growing in wells of micro-titer plates (96 well or 384 well) can be adapted to high through-put screening of libraries of compounds. Compounds in the library will be applied one at a time in an automated fashion to the wells of the microtitre dishes containing the transgenic cells described above. Once the test substances which activate one of the target genes are identified, it is preferable to then determine their site of action in the Integrated Stress Response pathway. It is particularly useful to define the site of action for the development of more refined assays for in order to optimize the target substance.

In some embodiments, the test substances that have been positively selected may be subjected to further selection steps in view of further assaying its properties in in vitro assays or in an animal model organism, such as a rodent animal model system, for the desired therapeutic activity prior to use in humans.

For example, in vitro assays may include use of T cell lines such as Jurkat cell line, or MOLT-4 cell line. In particular, the method may further comprise the steps consisting of providing a T cell line, bringing into contact the cell line with the selected test substance, determining the proliferation level of the T cell line, comparing said proliferation level with the proliferation level determined in the absence of the test substance, and positively selecting the test substance when the proliferation level determined in the presence of the test substance is lower that the proliferation level determined in the absence of the test substance. For example, assays which can be used to determine whether administration of a selected RASGRP1 inhibitor is indicated, include cell culture assays in which a patient tissue sample is grown in culture, and exposed to or otherwise contacted with a the RASGRP1 inhibitor, and the effect of such composition upon the tissue sample is observed. The tissue sample can be obtained by biopsy from the patient. This test allows the identification of the therapeutically most effective RASGRP1 inhibitor. In various specific embodiments, in vitro assays can be carried out with representative cells of cell types involved in an autoimmune (e.g., T cells), to determine if a test substance has a desired effect upon such cell types. Any well known animal model may be used for exploring the in vivo therapeutic effects of the screened RASGRP1 inhibitors. For example, the therapeutic activity of the screened RASGRP1 inhibitors can be determined by using various experimental animal models of inflammatory arthritis known in the art and described in Crofford L. J. and Wilder R. L., “Arthritis and Autoimmunity in Animals”, in Arthritis and Allied Conditions: A Textbook of Rheumatology, McCarty et al.(eds.), Chapter 30 (Lee and Febiger, 1993),. Experimental and spontaneous animal models of inflammatory arthritis and autoimmune rheumatic diseases can also be used to assess the anti-inflammatory activity of the screened RASGRP1 inhibitor. The effect of RASGRP1 inhibitors to reduce one or more symptoms of an autoimmune disease can be monitored/assessed using standard techniques known to one of skill in the art. Peripheral blood lymphocytes counts in a mammal can be determined by, e.g., obtaining a sample of peripheral blood from said mammal, separating the lymphocytes from other components of peripheral blood such as plasma using, e.g., Ficoll-Hypaque (Pharmacia) gradient centrifugation, and counting the lymphocytes using trypan blue. Peripheral blood T cell counts in mammal can be determined by, e.g., separating the lymphocytes from other components of peripheral blood such as plasma using, e.g., a use of Ficoll-Hypaque (Pharmacia) gradient centrifugation, labeling the T cells with an antibody directed to a T cell antigen such as CD2, CD3, CD4, and CD8 which is conjugated to FITC or phycoerythrin, and measuring the number of T cells by FACS. Further, the effect on a particular subset of T cells (e.g., CD2+, CD4+, CD8+, CD4+RO+, CD8+RO+, CD4+RA+, or CD8+RA+) cells can be determined using standard techniques known to one of skill in the art such as FACS. Thus the T cell proliferation in the animal model may be easily assessed. Other examples of animal models that can be used for the in vivo screening include animal for encephalomyelitis EAE, or 1 pr mice. The invention will be further illustrated by the following figures and examples.

However, these examples and figures should not be interpreted in any way as limiting the scope of the present invention.

### EXAMPLE

Material & Methods

Study approval. Informed and written consent was obtained from donors, patients and families of patients. The study and protocols are conform to the 1975 declaration of Helsinki as well as to local legislation and ethical guidelines from the Comite de Protection des Personnes de I'lle de France II and and the French advisory committee on data processing in medical research. Exome sequencing and analysis. Exome capture was performed according to the manufacturer's protocol using the Illumina TruSeq exome enrichment kit and sequencing of 100 bp paired end reads on an Illumina HiSeq. Approximately 10 Gb of sequence were obtained for each subject such that 90% of the coding bases of the exome defined by the consensus coding sequence (CCDS) project were covered by at least 10 reads. Adaptor sequences and quality trimmed reads were removed using the Fastx toolkit (http://hannonlab. cshl.edu/fastx_toolkit/) and a custom script was then used to ensure that only read pairs with both mates present were subsequently used. Reads were aligned to hg19 with BWA31, and duplicate reads were marked using Picard (http://picard.sourceforge.net/) and excluded from downstream analyses. Single nucleotide variants (SNVs) and short insertions and deletions (indels) were determined using samtools (http://samtools.sourceforge.net/) pileup and varFilter32 with the base alignment quality (BAQ) adjustment disabled, they were then quality filtered to require at least 20% of reads supporting the variant call. Variants were annotated using both ANNOVAR33 and custom scripts to identify whether they affected protein coding sequences, and whether they had previously been seen in the public data bases of exomes and the 7566 exomes previously sequenced at our center. The RASGRP1 variation identified in the patient (19:6586078G/A), a homozygous frameshift insertion c.1910_1911insAG p.Ala638Glyfs*Stop16 was not reported in the exome aggregation consortium (ExAC) database (http://exac.broadinstitute.org) nor in our institute database. It was not reported in other available public databases of exomes (dbSNP, the 1000 Genomes, the NHLBI Exome Sequencing Project (http://evs.gs.washington.edu/EVS/).

DNA sequencing. Genomic DNA from peripheral blood cells of the patient, their parents, and other family members was isolated according to standard methods. PCR products were amplified using Platinum Taq DNA Polymerase (Invitrogen) according to the manufacturer's recommendations, purified with the QlAquick gel extraction kit (Qiagen), sequenced using the ABI PRISM BigDye Terminator Cycle Sequencing Ready Reaction Kit (PerkinElmer) according to the manufacturer's recommendations and analyzed with 3500×L Genetic Analyzer (Applied Biosystems). All collected sequences were analyzed using DNADynamo (BlueTractorSoftware).

Gene expression analysis. Total RNA was isolated from T cell blasts of patient P1.1 and a control donor using the RNeasy Mini kit (QIAGEN). The samples were depleted of genomic DNA and reverse transcription was performed using Superscript II First Strand Synthesis System (Invitrogen). cDNAs were used as a template to perform PCR amplifications of the full RASGRP1 transcript. PCR products were verified by sequencing showing the expression of RASGRP1 mutated transcript (c.1910_191 linsAG) in patient cells.

Cell culture. Whole blood samples were collected from the patient and control donors. Peripheral blood mononuclear cells (PBMCs) were isolated by Ficoll-Paque density gradient (Lymphoprep, Proteogenix) from blood samples using standard procedures. Expansion of T-cell blasts were obtained by incubating PBMCs for 72 h with phytohaemagglutinin (PHA) (2.5 μg ml⁻¹, Sigma-Aldrich) in Panserin 401 (Pan Biotech) supplemented with 5% human male AB serum (BioWest), penicillin (100 U ml⁻¹) and streptomycin (100 μg ml⁻¹). After 3 days, dead cells were removed by Ficoll-Paque density gradient and blasts were maintained in culture with IL-2 (100 or 500 UI ml⁻¹). Before to be tested in the different assays, T-cell blasts were analyzed for CD3, CD4, CD8, CD45RO, CD45RA and CD57 expression. The phenotypes of T-cell blasts from healthy donors and the patient were comparable for the expression of these different markers.

Stimulation and proliferation assays. PHA-stimulated T cells were washed and cultured without IL-2 for 72 hours to synchronize the cells. Then PHA-stimulated T cells were cultured 4 days in complete medium alone or in the presence of 0.01, 0.1, 1 or 10 μg ml⁻¹ coated anti-CD3 antibody (clone OKT3, eBiosciences) (Invitrogen). Cell proliferation was monitored by labeling T cells with the CellTrace violet dye (Violet Proliferation Dye 450, BD Biosciences) prior to stimulation. After 4 days of culture, cells were harvested and CellTrace violet dye dilution was assessed by flow cytometry. Flow cytometry. Cell staining and the flow cytometry based phenotypic analyses of

PBMCs and cells were performed according to standard flow cytometry methods. The following monoclonal antibodies were conjugated to phycoerythrin-cyanin7 (PE-Cy7) Brilliant Violet 785 (BV785), Brilliant Violet 510 (BV510), Brilliant Violet (BV650), phycoerythrin (PE), phycoerythrin-cyanin5 (PE-Cy5), Brilliant Violet 451 (BV421), Peridinin-chlorophyll-cyanin5.5 (PerCP-Cy5.5): anti-CD25 (BC96), anti-CD3 (OKT3), anti-CD4 (OKT4), anti CD8 (RPA-T8), anti-CD27 (0323), anti-CD45RA (HI100), anti-CD161 (HP-3G10), anti-TCR Vα7.2 (3C10) all purchased from Sony Biotechnology Inc., anti-TCR Vα24 (C15), anti-TCR Vβ11 (C21), anti-TCR y6 (IMMU510) from Beckman Coulter and anti-CD19 (HIB19), anti-CD57 (NK-1) from BD biosciences. All data were collected on LSR-Fortessa cytometer (from BD Biosciences) and analyzed using FlowJo Version 10.0.8 software (Tree Star).

Cytokine production, degranulation and activation-induced cell death. For intracellular staining of cytokines, cells were stimulated overnight with coated anti-CD3 antibody, anti CD3/CD28 beads or PMA and ionomycine in the presence of brefeldin A (GolgiPlug, BD). Cells were then fixed and permeabilized using the BD cytofix/cytoperm plus kit (BD Pharmigen) according to the manufacturer's instructions. Cells were labeled with PE/Cy7-anti-TNF-α (mouse IgG1; Mab11) purchased from Sony Biotechnology Inc. and BV711-anti-IFN-γ (mouse IgG1; B27) from BD Biosciences or isotype-matched monoclonal antibodies. Cells were then analyzed by flow cytometry. Degranulation was determined by analysis of the expression of CD107/LAMP. Blasts were stimulated for 3 h in the presence of 0.1, 0,3, 1, 10 or 30 μg ml⁻¹ coated-OKT3 and simultaneously labelled with PE-anti-CD107a (H4A3) and PE-CD107b (H4B4) purchased from Sony Biotechnology Inc. Cells were then collected, washed and stained with BV785-anti-CD3, BV510-anti-CD4 and BV650-anti-CD8 monoclonal aibodies and analyzed by flow cytometry. Activation-induced cell death was examined 12 h after stimulation with coated anti-CD3 antibody by staining with 7-AAD Viaprobe (BD).

Immunoblotting. Cells (5.10⁶ cells per ml) were stimulated by anti-CD3 antibody (1 μg ml⁻¹) crosslinking with a donkey anti-mouse IgG (2 μg ml⁻¹) or anti-CD3+anti-CD28 coated beads (Invitrogen). Cells were then lysed in sample buffer. Proteins were separated by SDS-PAGE and transferred on PVDF membranes (Millipore). Membranes were blocked with milk or BSA for 1 h before incubation with primary antibodies. The following antibodies were used for immunoblotting: anti-phosphorylated tyrosine (4G10), anti-phosphorylated PLC-yl (#2821S), anti PLC-yl (#2822S), anti-phosphorylated ERK 1/2 (#4376S), anti ERK 1/2 (#4695S), anti-phosphorylated P38 (#4511S), anti phosphorylated AKT (Serine 473, 4058S) purchased from Cell Signaling Technology, anti-CTPS1 (EPR8086B) purchased from Abcam and anti-ACTIN (A2066) purchased from ThermoFischer Scientific, anti-RASGRP1 (#MABS146) from Merck Millipore and anti-PCNA (PC10) from Santa Cruz Technology. Membranes were then washed and incubated with anti-mouse or anti-rabbit HRP conjugated antibodies from Cell Signaling and GE Healthcare and Cell Signaling, respectively. Pierce ECL western blotting substrate was used for detection.

Calcium flux analysis. Blasts were loaded with 5 μM Indo-1 a.m (Molecular Probes), washed, incubated with APC-anti-CD4 and AF647-anti-CD8 antibodies. Cells were stimulated by anti-CD3 antibody (OKT3 5 μg/ml) crosslinking with F(ab′)2 rabbit anti-mouse IgG (10 μg/ml) and then incubated with ionomycin (1 μM). Cells were then analyzed with a FACSAria flow cytometer (BD Biosciences). Ca²⁺ flux data were obtained using kinetics analyses of FlowJo software package (TreeStar). Intracellular Ca²⁺ levels correspond to the normalized ratio of Ca²⁺-bound to Ca²⁺-free Indo-1 fluorescence and are plotted as a function of time.

Plasmids constructs, cell transfections and infections. A full-length cDNA encoding wild-type RASGRP1 was obtained by RT-PCR from control blasts. Full length cDNA encoding the mutant RASGRP1 was generated by mutagenesis using the Q5 Site-Directed Mutagenesis Kit (NEB). cDNAs were verified by sequencing, inserted into an expression vector pcDNA3.1D/V5-His-TOPO and transfected into HEK 293T cells using lipofectamine (Invitrogen). cDNAs were then also inserted into a bicistronic lentiviral expression vector encoding the green fluorescent prtein (GFP) as a reporter (pLenti7.3/V5-TOPO, Invitrogen). Viral particles for infection were obtained by co-expression of the lentiviral vector containing RASGRP1 with third-generation lentiviral plasmids containing Gag-Pol, Rev and the G protein of the vesicular stomatitis virus (VSVG) into HEK 293T. Viral supernatants were collected 48 h after transfection and viral particles were concentrated by ultracentrifugation. Control and patient's cells were infected with viral particles and the GFP expression was determined by flow cytometry.

Results and Discussion

Clinical Presentation and Immunogical Investigations

We studied two siblings of a single consanguineous family. Patients suffered from a exquisite susceptibility to EBV infection and Hodgkin lymphomas. The index case (P1.1) developed mixed cellularity EBV-positive Hodgkin lymphoma at the age of five years, treated by chemotherapy and autologous hematopoietic stem cell transplantation. He then had several episodes of EBV-triggered lymphoproliferation that were sensitive to anti-CD20 (Rituximab) administration. His sister (P1.2) developed at the age of 6 years a scleronodular EBV-positive Hodgkin lymphoma treated by chemotherapy. She had also an adrenal EBV smooth muscle tumor at the age of 7 years requiring surgery. She died at 11 years of age following relapse of Hodgkin lymphoma. Both patients also presented pneumonia and disseminated tuberculosis for P1.1 and Pneumocystis jiroveci pneumonia for P2.1. None of them had autoimmunity. Immunological investigations in P1.1 and P1.2 were carried out three and four years after chemotherapy, respectively. They revealed significant abnormalities including lymphocytopenia notably characterized by decreased counts of naïve CD4⁺ and CD8⁺ T cells, NK cells, MAIT and absence of iNKT cells, and impaired T-cell proliferation in response to PHA, OKT3 and Tetanus toxoid. The lymphocytopenia in P1.2 was more severe than in P1.1 possibly because of the lymphoma relapse at the time of the analysis. Serum immunoglobulin levels were normal or slightly increased. These observations strongly suggested that the immunodeficiency in two patients resulted from a T-cell immunodeficiency.

Identification of a Deleterious Mutation In RASGRP1

We performed whole-exome sequencing (WES) that identified 20 homozygous variations in patient P1.1. Only one of them appeared to be deleterious and was not found in public databases ExAc, 1000 genomes and in the database of our institute containing 8570 exomes. The identified mutation corresponds to a two nucleotides insertion in the exon 16 of the RASGRP1 gene (c.1910_1911insAG) leading to a frameshift resulting in a premature stop codon p.A1a638GlyfsX16 (or A638GfsStop16). The mutation was then verified by Sanger sequencing in the family. Both patients were homozygous for the mutation, while the two parents and the tested healthy sibling were heterozygous and wild-type carrier respectively, confirming the autosomal recessive inheritance mode of the mutation. The RASGRP1 codes for a diacylglycerol (DAG)-regulated guanidine exchange factor (GEF) highly and preferentially expressed in T and NK cells (Kortum et al., 2013). RASGRP1 is a specific activator the small G protein RAS through the exchange of RAS-bound GDP to GTP that in turn promotes activation of the Raf-MEK-ERK kinases cascade, which is essential for multiple cellular and developmental functions (Kortum et al., 2013). The premature stop codon in the mutant RASGRP1 protein is predicted to remove the entire C-terminal domain. RASGRP1 transcript expression in cells of P1.1 was found to be comparable to that of control cells. However, we failed to detect RASGRP1 protein expression in the lysate from P1.1, even when membranes were exposed for prolonged time (data not shown). In striking contrast, RASGRP1 was readily detected in lysates from healthy donors migrating as two species that likely differ by post translational modifications. Confirming the deleterious nature of the mutation, a faint band corresponding to mutated RASGRP1 was detectable in lysates from HEK 293 cells transiently transfected with a cDNA coding the mutant RASGRP1^{Ala638GlyfxX16}, whereas RASGRP1 was strongly expressed in lysates from HEK 293 cells transfected with wild-type RASGRP1.

Defective ERK1/2 in Activated RASGRP1-Deficient T Cells

Studies have demonstrated that RASGRP1 is required for T-cell antigen receptor (TCR)-mediated activation of the RAS-to-ERK pathway (Dower et al., 2000; Priatel et al., 2002). In human primary T cells, TCR-mediated ERK activation is mainly dependent of RASGRP1, although SOS1 and 2, two other GEFs expressed in lymphocytes have been shown to be also involved in RAS activation in T cells (Roose et al., 2005; Warnecke et al., 2012). We thus examined TCR-dependent signals in T-blasts from P1.1 and one healthy control upon TCR ligation by anti-CD3 antibody. In patient cells, global tyrosine phosphorylation of substrates of the TCR signaling cascade as well as Ca⁺⁺ mobilization were not really different from those seen in control cells after TCR stimulation, albeit Ca⁺⁺ mobilization appeared to be increased in T cells of the patient. Intriguingly, basal phosphorylation of PLC-γ1 before stimulation was found to be increased in patient T cells, possibly accounting for the enhanced Ca⁺⁺ flux observed in T cells of the patient. An increased level of DAG available for PLCγ-1 activation in absence of RASGRP1 might explain this phenomenon. Notably, when compared to control cells, phosphorylation of ERK1/2 kinases was found to be markedly reduced in patient cells. In comparison, p38 mitogen activated kinase and AKT kinase, that are not dependent of RAS were similarly phosphorylated in cells from the control and the patient as well. These results indicate that the pA638GfsStop16 mutation in RASGRP1 leads to a loss-of-protein expression resulting in defective activation of the RAS-to-ERK pathway in response to TCR stimulation.

Defective Proliferation of Activated RASGRPI-Deficient T Cells

RASGRP1-deficient null mice have been reported to exhibit a marked deficiency in development of mature thymocytes and lymphocytes that is associated with a lack of proliferation in response to TCR stimulation (Dower et al., 2000; Hogquist, 2001). Based on these findings and thus the recognized importance of the RAS pathway in cell proliferation, we analyzed in detail the proliferative capacity of T cells from the patient. When stimulated with an anti-CD3 antibody, P1.1 T cells weakly proliferated and failed to up regulate the activation marker CD25 when compared to control T cells that strongly divided and expressed CD25. In contrast, activation-induced cell-death and degranulation of P1.1 T cells in response to CD3 stimulation were found not to be significantly different from those of control cells, whereas TNF-α and IFN-γ production were moderately decreased and increased, respectively. Therefore, the defect in RASGRP1 preferentially results in impaired proliferation of activated lymphocytes in response to TCR.

In order to formally prove that the mutation in RASGRP1 is responsible for the impaired proliferation of RASGRP1-deficient T lymphocytes when activated through the TCR, we undertook complementation experiments in which wild-type RASGRP1 was introduced in T cells from P1.1 by using a lentiviral vector also containing a GFP reporter gene allowing to follow transduced cells. Control and patient T-cells were infected with empty or RASGRP1-containing constructs and then repeatedly stimulated with anti-CD3. Under theses conditions, GFP⁺ RASGRP1-deficient cells transduced with wild-type RASGRP1 exhibited a selective advantage and expanded in the culture. This was neither the case of RASGRP1-deficient cells that had been transduced with an empty vector nor control cells transduced with an empty or a wild type RASGRP1-containing vector, in which GFP⁺ cells had no advantage and did not accumulate. Taken together, these results show that expression of wild-type RASGRP1 in RASGRP1-deficient T cells restores their ability to proliferate and accumulate in response to TCR, thereby demonstrating the causal relationship between the RASGRP1 mutation and defective T-cell proliferation.

Defective CTPSI and PCNA Expression in Activated RAS-GRPI-Deficient T Cells

Interestingly, both the clinical phenotype and the defective T-cell proliferation associated with the RAS-GRP1 deficiency are reminiscent of the CTPS1-deficiency (Martin et al., 2014). Along these lines, we have previously shown that chemical inhibitors of ERK1/2 kinases inhibit CTPS1 up regulation in activated T cells, indicating that the RAS-to-ERK pathway is involved the expression of CTPS1. Hence, defective T-cell proliferation associated with RASGRP1 deficiency may be associated at least in part with a lack of up regulation of CTPS1 expression in response to TCR stimulation. We tested this possibility by analyzing CTPS1 expression in T cells from P1.1 following anti-CD3 stimulation. As previously reported, CTPS1 expression was up regulated after 12 hours of stimulation and persisted until 72 hours in control cells, whereas in activated P1.1 T cells only a slight and transient up regulation of CTPS1 was detectable after 12 hours of stimulation. These data confirm that CTPS1 expression is indeed dependent of RASGRP1. Defective CTPS1 expression in RASGRP1-deficient cells likely participates to the impaired proliferation capacity of these cells when activated by TCR. However, we observed that CTP or cytidine addition to the medium was not able to restore TCR-triggered proliferation of RASGRP1-deficient cells, in contrast to CTPS1-deficient T cells as we previously reported (Martin et al., 2014). Thus, this indicates that the RAS-ERK pathway exerts additional functions required for T-cell proliferation. In particular, other genes known to be involved in proliferation may be controlled by the RAS-ERK pathway. To examine this possibility, we tested the expression of the proliferating cell nuclear antigen PCNA, which plays a central role at the replication fork by recruiting enzymes required for DNA replication (Boehm et al., 2016). Similarly to CTPS1 expression, PCNA expression was found to be strongly decreased in patient activated T-cells. Further investigations are warranted to characterize in detail RASGRP1-dependent pathways that control T-cell proliferation.

We report herein a primary immunodeficiency resulting from a homozygous mutation in RASGRP1 that behaves as a loss-of-function mutation. Recent studies identified RASGRP1 has a locus for systemic lupus erythematosus susceptibility (Golinski et al., 2015). Autoimmunity was not noticed in both patients, but at a young age. Both patients had normal or slightly elevated immunoglobulins and develop EBV-driven B-cell lymphoproliferation, suggesting that RASGRP1-deficient B cells retained an intact ability to proliferate upon EBV transformation. By many aspects RASGRP1 deficiency phenotype resembles to CTPS1 deficiency. Like CTPS1, RASGRP1 appears to be critical for expansion of T cells that needs to be particularly intense and sustained during EBV infection (Hislop and Taylor, 2015; Taylor et al., 2015). This suggests that the defective proliferation capacity of antigen-driven T cells observed in both conditions is central in the impairment of immune response, in particular to EBV. RASGRP1 deficiency could in addition result in abnormalities of T-cell effector functions, like cytokine production as partially observed in P1.1. However, these abnormalities may only play a minor role as sustained T-cell expansion is an essential prerequisite to develop an efficient immune response to EBV (Hislop and Taylor, 2015; Taylor et al., 2015). The absence of iNKT cells found in P1.1 fits with the critical role of RASGRP1 in NKT cell development in mice (Shen et al., 2011). This cellular defect might also contribute to the impaired immune response to EBV infection in RASGRP1-deficient patients as iNKT cells have the ability to control of EBV-infected B cells and are often defective in primary deficiencies characterized by high susceptibility to EBV (Chung et al., 2013; Veillette et al., 2013). In conclusion, we report the first primary immunodeficiency caused by RASGRP1 deficiency associated with high susceptibility to EBV infection, underlining the critical role of RASGRP1 and the ERK pathway in anti-EBV immunity by their capacity to promote T-cell proliferation in response to antigenic stimulation.

### REFERENCES

Throughout this application, various references describe the state of the art to which this invention pertains. The disclosures of these references are hereby incorporated by reference into the present disclosure.

Boehm, E. M., M. S. Gildenberg, and M. T. Washington. 2016. The Many Roles of PCNA in Eukaryotic DNA Replication. Enzymes 39:231-254.

Chung, B. K., K. Tsai, L. L. Allan, D. J. Zheng, J. C. Nie, C. M. Biggs, M. R. Hasan, F. K. Kozak, P. van den Elzen, J. J. Priatel, and R. Tan. 2013. Innate immune control of EBV-infected B cells by invariant natural killer T cells. Blood 122:2600-2608.

Cohen, J. I. 2015. Primary Immunodeficiencies Associated with EBV Disease. Current topics in microbiology and immunology 390:241-265.

Dower, N. A., S. L. Stang, D. A. Bottorff, J. O. Ebinu, P. Dickie, H. L. Ostergaard, and J. C. Stone. 2000. RasGRP is essential for mouse thymocyte differentiation and TCR signaling. Nat Immunol 1:317-321.

Golinski, M. L., T. Vandhuick, C. Derambure, M. Freret, M. Lecuyer, C. Guillou, M. Hiron, O, Boyer, X. Le Loet, O. Vittecoq, and T. Lequerre. 2015. Dysregulation of RasGRP1 in rheumatoid arthritis and modulation of RasGRP3 as a biomarker of TNFalpha inhibitors. Arthritis Res Ther 17:382.

Hislop, A. D., and G. S. Taylor. 2015. T-Cell Responses to EBV. Current topics in microbiology and immunology 391:325-353.

Hogquist, K. 2001. RasGRP: the missing link for Ras activation in thymocytes. Trends Immunol 22:69.

Kortum, R. L., A. K. Rouquette-Jazdanian, and L .E. Samelson. 2013. Ras and extracellular signal-regulated kinase signaling in thymocytes and T cells. Trends Immunol 34:259-268.

Martin, E., N. Palmic, S. Sanquer, C. Lenoir, F. Hauck, C. Mongellaz, S. Fabrega, P. Nitschke, M. D. Esposti, J. Schwartzentruber, N. Taylor, J. Majewski, N. Jabado, R.F. Wynn, C. Picard, A. Fischer, P.D. Arkwright, and S. Latour. 2014. CTP synthase 1 deficiency in humans reveals its central role in lymphocyte proliferation. Nature 510:288-292.

Priatel, J. J., S. J. Teh, N. A. Dower, J. C. Stone, and H. S. Teh. 2002. RasGRP1 transduces low-grade TCR signals which are critical for T cell development, homeostasis, and differentiation. Immunity 17:617-627.

Roose, J. P., M. Mollenauer, V. A. Gupta, J. Stone, and A. Weiss. 2005. A diacylglycerol-protein kinase C-RasGRP1 pathway directs Ras activation upon antigen receptor stimulation of T cells. Mol Cell Biol 25:4426-4441.

Shen, S., Y. Chen, B.. Gorentla, J. Lu, J.. Stone, and X.. Zhong. 2011. Critical roles of RasGRP1 for invariant NKT cell development. J Immunol 187:4467-4473.

Taylor, G. S., H. M. Long, J. M. Brooks, A. B. Rickinson, and A. D. Hislop. 2015. The immunology of Epstein-Barr virus-induced disease. Annu Rev Immunol 33:787-821.

Veillette, A., L. A. Perez-Quintero, and S. Latour. 2013. X-linked lymphoproliferative syndromes and related autosomal recessive disorders. Current opinion in allergy and clinical immunology 13:614-622.

Warnecke, N., M. Poltorak, B. S. Kowtharapu, B. Arndt, J. C. Stone, B. Schraven, and L. Simeoni. 2012. TCR-mediated Erk activation does not depend on Sos and Grb2 in peripheral human T cells. EMBO Rep 13:386-391.

1. A method for inhibiting T cell proliferation in a subject in need thereof comprising administering to the subject a therapeutically effective amount of a RAS GRP1 inhibitor.

2. The method of claim 1 wherein the subject suffers from a T cell lymphoproliferative disease.

3. The method of claim 2 wherein the T cell lymphoproliferative disease is selected from the group consisting of lymphoblastic lymphomas; mature or peripheral T cell neoplasms, including T cell prolymphocytic leukemia, T-cell granular lymphocytic leukemia, aggressive NK-cell leukemia, cutaneous T cell lymphoma, anaplastic large cell lymphoma, T cell type lymphoma, enteropathy-type T cell lymphoma, Adult T-cell leukemia/lymphoma, angioimmunoblastic T cell lymphoma, subcutaneous panniculitic T cell lymphoma, and peripheral T cell lymphomas.

4. The method of claim 1 wherein the subject suffers from an autoimmune inflammatory disease.

5. The method of claim 4 wherein the autoimmune inflammatory disease is selected from the group consisting of arthritis, rheumatoid arthritis, acute arthritis, chronic rheumatoid arthritis, gouty arthritis, acute gouty arthritis, chronic inflammatory arthritis, degenerative arthritis, infectious arthritis, Lyme arthritis, proliferative arthritis, psoriatic arthritis, vertebral arthritis, and juvenile-onset rheumatoid arthritis, osteoarthritis, arthritis chronica progrediente, arthritis deformans, polyarthritis chronica primaria, reactive arthritis, ankylosing spondylitis, inflammatory hyperproliferative skin diseases, psoriasis, dermatitis, x-linked hyper IgM syndrome, urticaria, polymyositis/dermatomyositis, juvenile dermatomyositis, toxic epidermal necrolysis, scleroderma, systemic scleroderma, sclerosis, systemic sclerosis, multiple sclerosis (MS), spino-optical MS, primary progressive MS (PPMS), relapsing remitting MS (RRMS), progressive systemic sclerosis, atherosclerosis, arteriosclerosis, sclerosis disseminata, ataxic sclerosis, inflammatory bowel disease (IBD), Crohn's disease, colitis, ulcerative colitis, colitis ulcerosa, microscopic colitis, collagenous colitis, colitis polyposa, necrotizing enterocolitis, transmural colitis, autoimmune inflammatory bowel disease, pyoderma gangrenosum, erythema nodosum, primary sclerosing cholangitis, episcleritis, respiratory distress syndrome, adult or acute respiratory distress syndrome (ARDS), meningitis, inflammation of all or part of the uvea, iritis, choroiditis, an autoimmune hematological disorder, rheumatoid spondylitis, sudden hearing loss, an IgE-mediated disease, encephalitis, Rasmussen's encephalitis, limbic and/or brainstem encephalitis, uveitis, anterior uveitis, acute anterior uveitis, granulomatous uveitis, nongranulomatous uveitis, phacoantigenic uveitis, posterior uveitis, autoimmune uveitis, glomerulonephritis (GN), idiopathic membranous GN, idiopathic membranous nephropathy, membrano- or membranous proliferative GN (MPGN), rapidly progressive GN, allergic conditions, autoimmune myocarditis, leukocyte adhesion deficiency, systemic lupus erythematosus (SLE), systemic lupus erythematodes cutaneous SLE, subacute cutaneous lupus erythematosus, neonatal lupus syndrome (NLE), lupus erythematosus disseminatus, lupus, juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, immune responses associated with acute and delayed hypersensitivity mediated by cytokines and T-lymphocytes, tuberculosis, sarcoidosis, granulomatosis, lymphomatoid granulomatosis, Wegener's granulomatosis, agranulocytosis, vasculitides, vasculitis, large vessel vasculitis, polymyalgia rheumatica, giant cell (Takayasu's) arteritis, medium vessel vasculitis, Kawasaki's disease, polyarteritis nodosa, microscopic polyarteritis, CNS vasculitis, necrotizing, cutaneous, hypersensitivity vasculitis, systemic necrotizing vasculitis, ANCA-associated vasculitis, Churg-Strauss vasculitis or syndrome (CSS), temporal arteritis, aplastic anemia, autoimmune aplastic anemia, Coombs positive anemia, Diamond Blackfan anemia, hemolytic anemia, immune hemolytic anemia, autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome, antigen-antibody complex-mediated diseases, anti-glomerular basement membrane disease, anti-phospholipid antibody syndrome, allergic neuritis, Bechet's or Behcet's disease, Castleman's syndrome, Goodpasture's syndrome, Reynaud's syndrome, Sjogren's syndrome, Stevens-Johnson syndrome, pemphigoid, pemphigoid bullous, skin pemphigoid, pemphigus, pemphigus vulgaris, pemphigus foliaceus, pemphigus mucus-membrane pemphigoid, pemphigus erythematosus, autoimmune polyendocrinopathies, Reiter's disease or syndrome, immune complex nephritis, antibody-mediated nephritis, neuromyelitis optica, polyneuropathies, chronic neuropathy, IgM polyneuropathies, IgM-mediated neuropathy, thrombocytopenia, thrombotic thrombocytopenic purpura (TTP), idiopathic thrombocytopenic purpura (ITP), autoimmune orchitis, autoimmune oophoritis, primary hypothyroidism, hypoparathyroidism, autoimmune thyroiditis, Hashimoto's disease, chronic thyroiditis (Hashimoto's thyroiditis), subacute thyroiditis, autoimmune thyroid disease, idiopathic hypothyroidism, Grave's disease, polyglandular syndromes, autoimmune polyglandular syndromes, polyglandular endocrinopathy syndromes, paraneoplastic syndromes, neurologic paraneoplastic syndromes, Lambert-Eaton myasthenic syndrome, Eaton-Lambert syndrome, stiff-man or stiff-person syndrome, encephalomyelitis, allergic encephalomyelitis, experimental allergic encephalomyelitis (EAE), myasthenia gravis, thymoma-associated myasthenia gravis, cerebellar degeneration, neuromyotonia, opsoclonus or opsoclonus myoclonus syndrome (OMS), sensory neuropathy, multifocal motor neuropathy, Sheehan's syndrome, autoimmune hepatitis, chronic hepatitis, lupoid hepatitis, giant cell hepatitis, chronic active hepatitis, autoimmune chronic active hepatitis, lymphoid interstitial pneumonitis, bronchiolitis obliterans, Guillain-Barre syndrome, Berger's disease nephropathy), idiopathic IgA nephropathy, linear IgA dermatosis, primary biliary cirrhosis, pneumonocirrhosis, autoimmune enteropathy syndrome, Celiac disease, Coeliac disease, celiac sprue, refractory sprue, idiopathic sprue, cryoglobulinemia, amylotrophic lateral sclerosis (ALS), coronary artery disease, autoimmune ear diseas, autoimmune inner ear disease (AGED), autoimmune hearing loss, opsoclonus myoclonus syndrome (OMS), polychondritis, pulmonary alveolar proteinosis, amyloidosis, scleritis, a non-cancerous lymphocytosis, a primary lymphocytosis, MGUS, peripheral neuropathy, paraneoplastic syndrome, channelopathies, autism, inflammatory myopathy, focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases, autoimmune demyelinating diseases, diabetic nephropathy, Dressler's syndrome, alopecia greata, CREST syndrome telangiectasia, male and female autoimmune infertility, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis, allergic alveolitis and fibrosing alveolitis, interstitial lung disease, transfusion reaction, leprosy, malaria, leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis, chronic cyclitis, heterochronic cyclitis, iridocyclitis, or Fuch's cyclitis, Henoch-Schonlein purpura, human immunodeficiency virus (HIV) infection, echovirus infection, cardiomyopathy, Alzheimer's disease, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, endocrine ophthamopathy, chronic hypersensitivity pneumonitis, keratoconjunctivitis sicca, epidemic keratoconjunctivitis, idiopathic nephritic syndrome, minimal change nephropathy, benign familial and ischemia-reperfusion injury, retinal autoimmunity, joint inflammation, bronchitis, chronic obstructive airway disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired splenic atrophy, infertility due to antispermatozoan antobodies, non-malignant thymoma, vitiligo, SCID, Epstein-Barr virus-associated diseases, acquired immune deficiency syndrome (AIDS), parasitic diseases, Lesihmania, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, peripheral neuropathy, autoimmune polyglandular syndrome type I, adult-onset idiopathic hypoparathyroidism (AOIH), alopecia totalis, dilated cardiomyopathy, epidermolisis bullosa acquisita (EBA), hemochromatosis, myocarditis, nephrotic syndrome, primary sclerosing cholangitis, sinusitis, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia, autoimmune disorders associated with collagen disease, rheumatism, neurological disease, ischemic re-perfusion disorder, reduction in blood pressure response, vascular dysfunction, antgiectasis, tissue injury, cardiovascular ischemia, hyperalgesia, cerebral ischemia, and disease accompanying vascularization, allergic hypersensitivity disorders, glomerulonephritides, reperfusion injury, reperfusion injury of myocardial tissues, dermatoses with acute inflammatory components, acute purulent meningitis, ocular and orbital inflammatory disorders, granulocyte transfusion-associated syndromes, cytokine-induced toxicity, acute serious inflammation, chronic intractable inflammation, pyelitis, pneumonocirrhosis, diabetic retinopathy, diabetic large-artery disorder, endarterial hyperplasia, peptic ulcer, valvulitis, and endometriosis.

6. The method of claim 1 wherein the subject suffers from an allergic disorder.

7. The method of claim 1 wherein the subject is a transplant recipient.

8. The method of claim 7 wherein the transplant recipient has received a transplanted graft selected from the group consisting of heart, kidney, lung, liver, pancreas, pancreatic islets, brain tissue, stomach, large intestine, small intestine, cornea, skin, trachea, bone, bone marrow, muscle, and bladder.

9. The method of claim 7 wherein the step of administering prevents or suppresses an immune response associated with rejection of a donor tissue, cell, graft, or organ transplant by a the transplant recipient.

10. The method of claim 7 wherein the transplant recipient suffers from graft versus host disease (GVDH) or Host-Versus-Graft-Disease.

11. A method for screening a plurality of test substances useful for inhibiting T cell proliferation in a subject in need thereof comprising the steps consisting of i) testing each of the test substances for its ability to inhibit RASGRP1 activity or expression and ii) identifying the test substance which inhibits RASGRP1 activity or expression as a test substance useful for inhibiting T cell proliferation in a subject in need thereof.

12. The method of claim 5, wherein the psoriasis is plaque psoriasis, gutatte psoriasis, pustular psoriasis, or psoriasis of the nails.

13. The method of claim 5, wherein the dermatitis is contact dermatitis, chronic contact dermatitis, allergic dermatitis, allergic contact dermatitis, dermatitis herpetiformis, or atopic dermatitis.

14. The method of claim 5, wherein the urticaria is chronic allergic urticarial, chronic idiopathic urticaria, or chronic autoimmune urticarial.

15. The method of claim 5, wherein the IgE-mediated disease is anaphylaxis, allergic rhinitis and atopic rhinitis.

16. The method of claim 5, wherein the multiple organ injury syndrome is secondary to septicemia, trauma or hemorrhage.

17. The method of claim 5, wherein the channelopathy is epilepsy, migraine, arrhythmia, muscular disorders, deafness, blindness, periodic paralysis, or a channelopathy of the central nervous system (CNS).

18. The method of claim 5, wherein the alveolitis is allergic alveolitis or fibrosing alveolitis.

19. The method of claim 5, wherein the cyclitis is chronic cyclitis, heterochronic cyclitis, iridocyclitis, or Fuch's cyclitis.

20. The method of claim 5, wherein the sinusitis is purulent sinusitis, nonpurulent sinusitis, acute sinusitis, chronic sinusitis, ethmoid sinusitis, frontal sinusitis, maxillary sinusitis, or sphenoid sinusitis.

21. The method of claim 5, wherein the eosinophil-related disorder is eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils.
